(19) United States
(12) Patent Application Publication
(10) Pub. No.: US 2009/0281623 A1
(43) Pub. Date: Nov. 12, 2009

(54) CUSTOMIZATION OF IMPLANTABLE MEDICAL DEVICES

(75) Inventors: John E. Kast, Hugo, MN (US); Mark D. Wagy, Minneapolis, MN (US); Micah A. Litow, Minneapolis, MN (US)

Correspondence Address: SHUMAKER & SIEFFERT , P.A, 1625 RADIO DRIVE , SUITE 300, WOODBURY, MN, 55125, US

(73) Assignee: Medtronic, Inc.

(21) Appl. No.: 12/119,044

(22) Filed: May 12, 2008

(51) Int. Cl. A61F 2/02

(52) U.S. Cl. 623/11.11

(57) ABSTRACT

A custom implantable medical device (IMD) may be formed based on an image of a structure of a patient, such as the head of the patient. The custom implantable medical device may include a custom member that at least partially encapsulates a module of the IMD, or may include a custom housing that encloses a module of the IMD. The custom member or custom housing may be constructed to conform to a shape determined based on the image. The image of the head of the patient may include an image of a skull of the patient, an image of a scalp of the patient, or an image of vascular or neurological structures in the head of the patient.

### TECHNICAL FIELD

The disclosure relates to medical devices, and more particularly, to implantable medical devices that deliver therapy to and/or monitor a patient.

### BACKGROUND

Implantable medical devices (IMDs) include devices implantable in a human or other animal body that sense medical parameters, monitor medical conditions, administer therapy, or any combination thereof. Typical IMDs include a variety of electrical and/or mechanical components, and may include a housing that houses the components. Because the components may be fragile, the housing is usually sufficiently robust to protect the components from forces to which they would otherwise be exposed when implanted within the body. Housings may be constructed from titanium or a titanium alloy, for example. In order to avoid potentially harmful interactions between the components and bodily fluids, such as corrosion, IMD housings are typically hermetically sealed.

Large components common to most IMDs typically include a battery, a coil, and a hybrid circuit that includes digital circuits, e.g., integrated circuit chips and/or a microprocessor, and analog circuit components. IMDs may include other components as well. The components and the housing each add bulk to the IMD.

Some medical devices may be implanted in the head of a patient. For example, an IMD may be implanted under the scalp and on top of the cranium, with one or more leads deployed on the head or implanted in the brain.

### SUMMARY

In general, the disclosure is directed to techniques for determining a shape of an IMD based on an image of a head of a patient and constructing the IMD such that an exterior surface of the IMD conforms to the shape. In some examples, the IMD may be configured to be implanted under the scalp of a patient and on top of the skull of the patient.

Generally, an image is collected prior to surgery, based on which a shape of the IMD is determined. The collected image can pertain to the contours of the skull of the patient, the condition of the scalp of the patient, the vascular structure or neurological structures in the head of the patient, and the like. The image may be generated by X-ray, magnetic resonance imaging, CT-scan and fluoroscopy. The image can be represented as a physical or a virtual model of the skull of the patient and the IMD.

An exterior surface (or surfaces) of the IMD is then constructed to conform to the shape determined based on the image. For example, a custom member, which at least partially encapsulates a module of the IMD, may be constructed to conform to the shape. In some examples, the IMD includes a hermetic housing that encloses the module and the custom member at least partially encapsulates the housing, while in other examples, the custom member may be coated with a hermetic coating. As another example, a custom housing may be constructed to conform to the shape.

In one aspect, the disclosure is directed to a method comprising receiving an image of tissue of a patient, determining a shape of a custom member that at least partially encapsulates a module of an implantable medical device based on the image, the module comprising control electronics and constructing the custom member to conform to the shape.

In another aspect, the disclosure is directed to a method including receiving an image of tissue of a patient, determining a shape of a custom housing of an implantable medical device comprising control electronics based on the image, and constructing the custom housing to conform to the shape.

In a further aspect, the disclosure is directed to an implantable medical device including a module comprising control electronics, a custom member that at least partially encapsulates the module, and a hermetic coating formed over the custom member. The custom member is constructed to conform to a shape determined based on an image of tissue of a patient in which the implantable medical device is to be implanted.

In an additional aspect, the disclosure is directed to an implantable medical device comprising a custom housing for housing a module comprising control electronics constructed to conform to a shape determined based on an image of tissue of a patient in which the implantable medical device is to be implanted.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example deployment of an IMD under the scalp of a patient.

FIG. 2 is a plan diagram of the top of a head of a patient, illustrating an example implantation of an IMD.

FIG. 3 is a flow diagram illustrating an example method of determining a shape and constructing an IMD based on image data of a head of a patient.

FIG. 4 is a conceptual diagram illustrating an example use of image data to develop physical or virtual models.

FIG. 5 is a flow diagram illustrating another example method of determining a shape and constructing an IMD based on image data of a head of a patient.

FIG. 6 is a flow diagram illustrating an example method of constructing a custom member.

FIGS. 7A and 7B are conceptual diagrams illustrating an IMD including a custom member before and after machining.

FIG. 8 is a flow diagram illustrating another example method of constructing a custom member.

FIG. 9 is a flow diagram illustrating an example method of constructing a custom member including a hermetic coating.

FIGS. 10A-10C are conceptual diagrams illustrating a method of constructing an IMD including a custom member comprising a hermetic coating.

FIG. 11 is a conceptual diagram of an example IMD that includes a custom housing.

FIG. 12 is a plan diagram of one example of an IMD that includes a custom grippable access structure in the form of a loop.

FIG. 13 is a plan diagram of another example of an IMD that includes a custom grippable access structure in the form of a tab.

FIG. 14 is a perspective view of an example of an IMD that includes a custom lead management structure.

### DETAILED DESCRIPTION

When an implantable medical device (IMD) is implanted under a scalp, but over or recessed into the skull, of a patient, it may be desirable to minimize the size (e.g., volume) of the IMD to minimize adverse physical and cosmetic consequences of the implantation. For example, a larger IMD or an IMD with sharp edges may erode the scalp of the patient, and may also produce an unsightly bump in the scalp of the patient. However, reducing the size of the IMD may require performance compromises, such as reduced battery life.

Further, IMDs may be implanted in a wide variety of patients, who may have different head sizes or head shapes (e.g., cranium sizes or shapes). In addition, a surgeon may wish to implant the IMD in a different location for some patients due to, for example, a different therapy location, or features of the head of the patient that preclude implantation of the IMD at another location. This may lead to further complications when implanting a standard IMD, because the IMD may not conform to the shape of the skull at the implant location, which may exacerbate the physical and cosmetic consequences of the implantation.

One method of addressing these consequences may include constructing a custom IMD or an IMD including at least a custom outer surface. The custom IMD or custom outer surface of the IMD may alleviate the stress on the scalp of the patient, and may also improve the cosmetic appearance of the implant by smoothing the transition from an area of the scalp with no implant to an area of the scalp with an IMD implanted underneath. Described herein are novel techniques of constructing a custom IMD including a shape determined based on an image of a head of a patient into which the IMD is to be implanted. Constructing an IMD in this manner may reduce adverse physical and cosmetic consequences of implanting an IMD on top of a skull of a patient. In some examples, the IMD comprises a custom member that at least partially encapsulates a module of the IMD. The custom member is constructed to conform to the shape. In other examples, the IMD may comprise a custom housing that is constructed to conform to the shape.

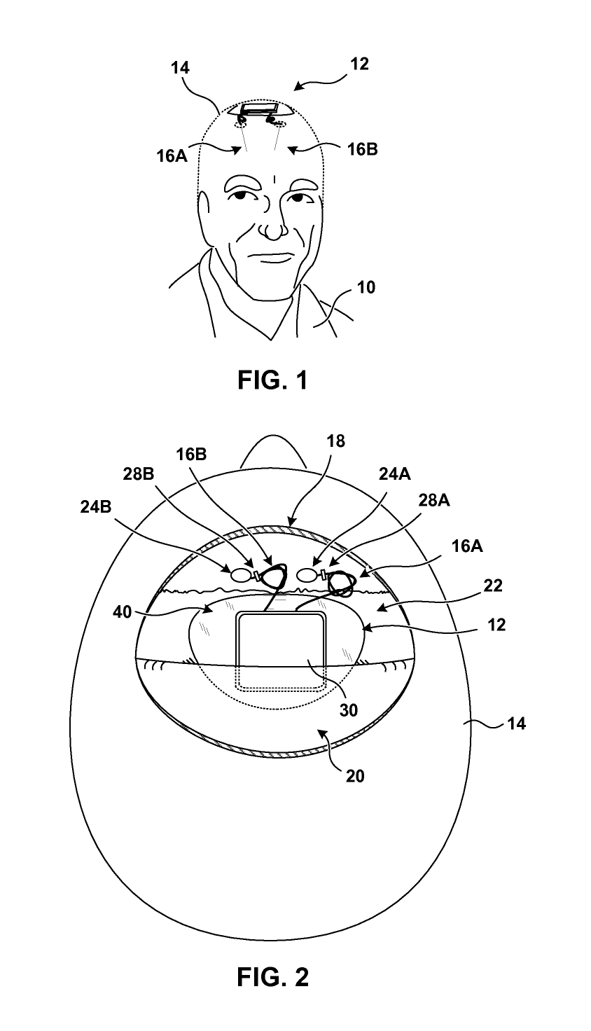

FIG. 1 shows a patient 10 with an IMD 12 deployed beneath his scalp 14. In some examples, IMD 12 may be a low-profile device, i.e., may have a relatively small cross-sectional height. In FIG. 1, IMD 12 is a neurostimulator that provides deep brain stimulation via leads 16A and 16B deployed in the brain of patient 10. In the example of FIG. 1, IMD 12 is deployed in proximity to the site of stimulation therapy. IMD 12 may be used to treat any nervous system disorder including, but not limited to, epilepsy, pain, psychological disorders including mood and anxiety disorders, movement disorders (MVD) such as, but not limited to, essential tremor and Parkinson's disease and neurodegenerative disorders.

Although IMD 12 is depicted as a neurostimulator, the techniques of the disclosure are not limited to applications in which the IMD is a neurostimulator. The techniques of the disclosure may be employed with IMDs that perform any monitoring or therapeutic functions. The techniques of the disclosure are not limited to IMDs that include leads deployed in the brain, but may also be employed with leads deployed anywhere in the head or neck including, for example, leads deployed on or near the surface of the skull, leads deployed beneath the skull such as near or on the dura mater, leads placed adjacent cranial or other nerves in the neck or head, or leads placed directly on the surface of the brain. Nor are the techniques of the disclosure limited to IMDs that are coupled to electrodes. The techniques of the disclosure may be employed with IMDs coupled to any sensing or therapeutic elements, such as temperature sensors or motion sensors. The techniques of the disclosure may also be employed with different types of IMDs including, but not limited to, IMDs operating in an open loop mode (also referred to as non-responsive operation), IMDs operating in a closed loop mode (also referred to as responsive), and IMDs for providing monitoring and/or warning.

In the example of FIG. 1, IMD 12 is deployed beneath scalp 14 of patient 10, but on top of the cranium of patient 10. The techniques of the disclosure may be applied to other types of implantation as well, such as implantation of at least a part of IMD 12 in a trough or recess cut into the cranium of patient 10. Further, the techniques of the disclosure are not limited to IMDs implanted in a head of a patient. For example, the techniques of the disclosure may be employed with IMDs, such as implantable pulse generators (IPGs) or implantable cardiac defibrillators (ICDs) which provide stimulation therapy to a heart of a patient. Additionally, the techniques of the disclosure may be employed with IMDs that perform any monitoring or therapeutic functions in other locations within a body of a patient.

IMD 12 may comprise a shape determined based on an image of patient 10, e.g., of the head of the patient in the case of the examples described herein. The image may be generated by, for example, an X-ray, CT-scan, magnetic resonance imaging, or fluoroscopy, and may include an image of bone and/or soft tissue, e.g., a skull and/or a scalp. The image may be provided to a processor of a computer system, and the processor may produce a representation of the head. The processor may determine the shape based on the representation of the head to, for example, improve the conformity of a shape of IMD 12 to the head of patient 10. In some examples, at least a portion of the shape of IMD 12 may be defined by a custom member that at least partially encapsulates a module of IMD 12. In other examples, the shape of IMD 12 may be defined by a custom housing. The custom member or custom housing may be constructed to conform to the determined shape, and may be delivered to a clinic, clinician, or surgeon for implantation in patient 10 after construction to conform to the determined, patient-specific shape.

FIG. 2 illustrates a typical implantation of IMD 12 shown in FIG. 1. In a typical implantation, the surgeon makes an incision 18 through the scalp 14 of patient 10, and pulls back the resulting flap of skin 20 to expose the desired area of the cranium 22. The incision may be a “C-flap” incision, for example. Patient 10 may be under local anesthetic. The surgeon drills holes, called “burr holes,” in the cranium and deploys leads 16 through the burr holes into the brain.

The surgeon typically places caps 24A and 24B, called “burr hole caps,” over the burr holes. A portion of each of the bodies of leads 16A and 16B is deployed outside of the brain on the surface of skull 22. Before connecting leads 16A and 16B to IMD 12, the surgeon may “manage” the leads. Lead management may include arranging the excess length of leads 16 using techniques such as coiling and anchoring with anchoring plates. In a typical implantation, the surgeon arranges the leads to provide some slack to reduce the risk of lead migration. In FIG. 2, leads 16A and 16B are depicted as coiled, and are anchored by anchoring plates 28A and 28B.

In some examples, the custom member or custom housing of IMD 12 may include custom lead management features, such as a channel or cavity, as described in further detail below. For example, the surgeon may have a preference for one method of lead management compared to a second method of lead management. The surgeon may indicate this preference to a manufacturer of IMD 12, and the manufacturer may construct IMD 12 to include the surgeon's preferred lead management structure.

The surgeon implants IMD 12 between scalp 14 and skull 22. In one surgical procedure, the surgeon uses a tool to form a pocket beneath scalp 14 proximate to the burr holes, and positions IMD 12 in the pocket. The surgeon may fix IMD 12 to the cranium using an attachment mechanism such as bone screws. The surgeon closes skin flap 20 over IMD 12, and then staples or sutures the incision.

In FIG. 2, IMD 12 may be a low-profile device including a custom member or custom housing, allowing it to be implanted between scalp 14 and skull 22 with little discomfort or adverse cosmetic consequences to patient 10. A low-profile IMD also typically lacks high protrusions with sharp corners that could cause skin erosion. IMD 12 comprises one or more modules that carry out the various functions of IMD 12. In the example illustrated by FIG. 2, IMD 12 may include a single module 30, but in other examples may include two or more separate modules, which may provide different functionality or include different components of IMD 12.

Module 30 may include a housing that can carry out a variety of functions, including encasing the components of module 30, sealing the module against contamination (e.g., a hermetic seal), electrically isolating electrical components, and the like. Module 30 may include any of a variety of components within the housing, such as a processor or other control electronics, other digital or analog circuitry, a power source such as a rechargeable or nonrechargeable battery, communication circuitry, an antenna, electrical stimulation generation circuitry, a pump, a reservoir, or physiological parameter sensing circuitry. In other examples in which the IMD includes multiple modules, such modules may also include a housing and house some portion of these components, e.g., a separate module for housing a power source of the IMD.

In the example illustrated in FIG. 2, module 30 is coupled to a member 36, which may be made of a biocompatible material. Member 36 at least partially encapsulates module 30, and generally serves as a smooth interface between the modules and the body tissue. Member 36 may comprise a custom shape determined based on an image of the head of patient 10, which may include an image of skull 22, scalp 14, or other soft tissue, as will be described in further detail below.

In general, member 36 may be flexible or relatively rigid. Member 36 may be made from silicone, polyurethane, polysulfone, epoxy, stainless steel, titanium, an alloy, such as, for example, a titanium alloy, or the like, and in some examples may be made from two or more materials of differing flexibility, such as silicone and a polyurethane. Member 36 may be molded, machined, or may be formed by another process.

The IMD is not limited to the particular IMD 12 depicted in FIG. 2, but includes a number of embodiments, some of which are described in more detail below. For example, the IMD may not include a member that at least partially encapsulates the at least one module, and may instead include a custom housing. The custom housing may comprise a drawn metal, where the metal is drawn over a custom form. In some examples, the custom housing comprises stainless steel, titanium, or an alloy, such as, for example, a titanium alloy, and may provide a hermetic seal around the at least one module.

In other examples, IMD 12 may include one module or two modules, and the modules may or may not include a housing. In some examples, the one or two modules may include a module that includes both a recharge module and power supply module, both a power supply module and control module, or another combination of modules. Member 36 may at least partially encapsulate each of the modules, and in some examples may fully encapsulate one or more of the modules. In some examples, as described below, member 36 may be coated with a hermetic coating, such as titanium, a titanium alloy, or gold.

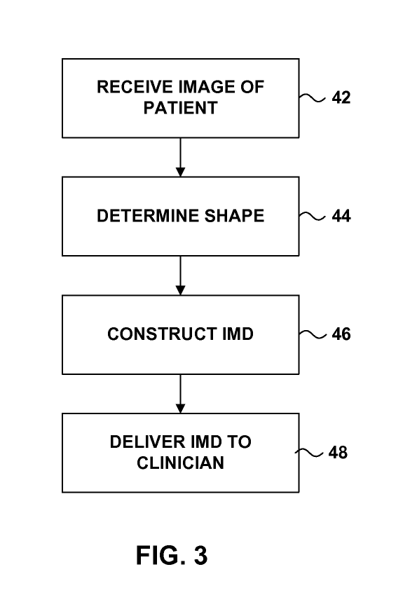

FIG. 3 is a flow diagram of an example method by which a shape of an IMD is determined and the IMD constructed to conform to the shape. According to the example method of FIG. 3, an imaging apparatus, such as, for example, an X-ray, magnetic resonance imaging, CT-scan, or fluoroscopy apparatus, collects an image of a patient, e.g., of a head of a patient, and transmits the image to a computer system, which receives the image (40). The image may include an image of a skull, scalp, or other soft tissue, or may be a composite image including, for example, both the skull and scalp. The image includes data that may be used to construct a virtual or physical model of the head, or a portion of the head, or some other portion or structure of patient. Where the model is of the head, the module may include information including contour and thickness of the skull, a condition of the scalp, locations of vascular and neurological structures, and the like.

The computer system, either with input from a surgeon or technician, or automatically, determines a shape of the IMD (44). The shape may include the shape of one or more surfaces of the IMD, including, for example, the shape of a surface adjacent the skull of the patient or a surface adjacent the scalp of the patient.

A manufacturing system then constructs an IMD to conform to the shape (46). As described in further detail below, the IMD may include a custom member that at least partially encapsulates at least one module of the IMD. The custom member may be constructed to conform to the shape. In some examples, the module includes a housing, and the custom member at least partially encapsulates the housing. In other examples, the custom member at least partially, and may fully, encapsulate the module, and a hermetic coating may be coated over the custom member. In yet other examples, the IMD may not include a custom member, and instead includes a custom housing constructed to conform to the shape, which encloses the module. The IMD constructed to conform to the shape may then be delivered to a clinician, e.g., surgeon, for implantation as a patient-specific, custom IMD.

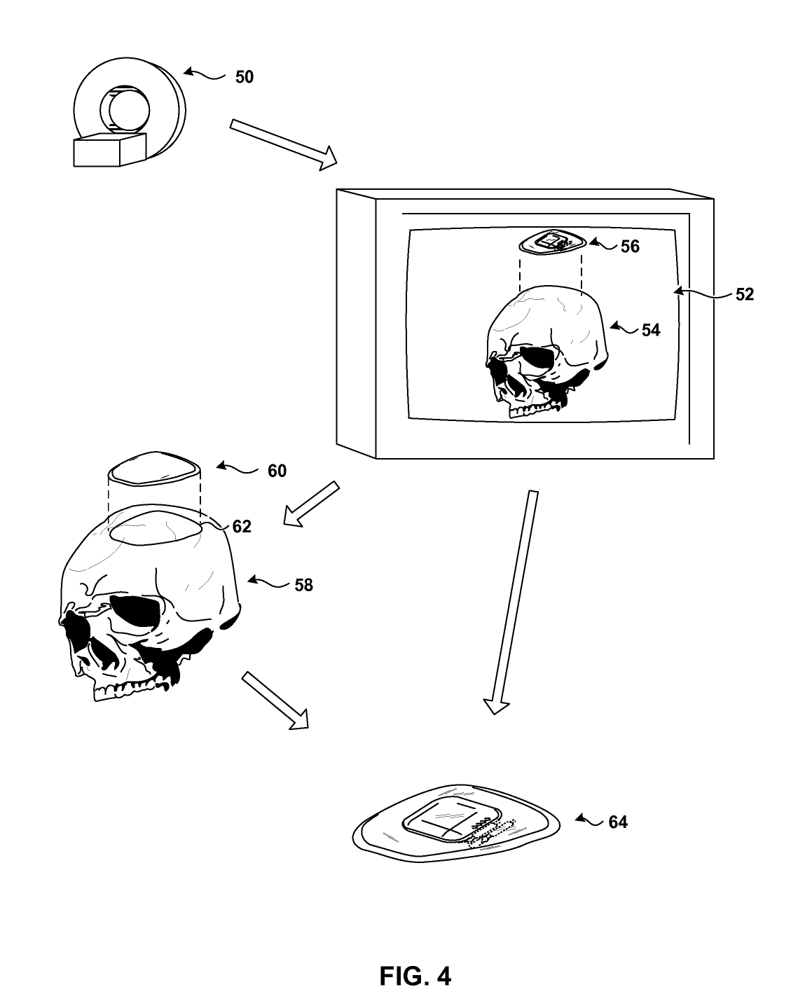

FIG. 4 is a conceptual diagram showing an example system for determining a shape of an IMD to be implanted between a scalp and a skull of a patient and constructing the IMD to conform to the shape. One or more pieces of imaging equipment 50 collects images of the skull of the patient, the scalp of the patient, or other soft tissue, such as vascular or neurological structures, resulting in image data. The image data may include images generated by X-ray, magnetic resonance imaging, CT-scan and fluoroscopy.

The image data are supplied to a computer system 52, which may construct a virtual model 54 of the skull of the patient. In some examples, the virtual model 54 may be used to construct a physical model 58 of the skull of the patient. In other examples, the virtual model 54 is used, along with a virtual IMD 56, to determine the shape of physical IMD 64.

In one example, a physical model 58 of the skull of the patient is constructed based on the image data. Physical model 58 is a representation of the skull of the patient. In some examples, a moldable material 60 may be tailored to conform to a region 62 of the physical model 58. The region 62 may be determined based on the feasibility of implantation of physical IMD 64 at this location. The feasibility of implantation at a region may be determined based on, for example, a shape or thickness of the skull of the patient in this location, presence of vascular structures at this location, necessity of forming a recess in the skull of the patient, or proximity to the location of a burr hole. Once the desired region 62 is determined, and any modification made to the region 62, such as forming a recess in the physical model 58, the moldable material 60 is molded against the region 62. For example, the moldable material 60 may comprise a material with a softening temperature that allows it to be heated, pressed against the physical model 48, worked into a desired shape, and cooled below the softening temperature and allowed to harden, resulting in a shape that conforms to the region 62 of the physical model 58 corresponding to the location of the skull of patient 11 at which physical IMD 64 is to be implanted.

In various examples, the moldable material 60 may be used as a form over which a custom housing is drawn, may be used to make a mold against which a custom member is molded, or may be the custom member.

In some examples, the moldable material 60 may subsequently used to create a mold insert against which a custom member is molded. After being pressed against the physical model 58, the moldable material 60 may comprise a negative impression of the region 62. The moldable material 60 may then be used to construct the mold insert, which comprises a negative impression of the moldable material 60, or a replica of the region 62. The mold insert may then be used in a molding process, such as injection molding, compression molding, reactive injection molding, or the like, to form a custom member that conforms to the shape of the mold insert, and thus conforms to the shape of the region 62 of the skull against which the physical IMD 64 is to be implanted.

In other examples, a section of physical model 58 may be used as the mold insert. For example, a section of the physical model 58 including the region 62 may be removed from the physical model 58. This section may be used as the mold insert, and a custom member may be formed by molding a material against the section in an injection molding machine, a compression molding machine, or the like.

In yet other examples, the moldable material 60 may be used as a form over which a metal is drawn. For example, the moldable material 60 may be molded against region 62, allowed to harden, and then the other surfaces (e.g., the surface which did not contact region 62) may be worked into a desired shape of an IMD. The hardened moldable material 60 may then be used as a form over which a custom housing may be drawn, in examples in which the hardened moldable material 60 comprises sufficient strength. In other examples, the hardened moldable material 60 may be used to create a mold, which may then be used to create a form, which is then used to construct the custom housing by drawing a metal, such as titanium or a titanium alloy, over the form.

In another example, the computer system 52 generates a virtual model 54 of the patient skull and a virtual IMD 56. In some examples, virtual IMD 56 may be selected from a plurality of virtual IMD starting configurations and can then be manipulated to conform to virtual skull 54. For example, the plurality of virtual IMD starting configurations may include different configurations of at least one module within a member, and may also include a virtual IMD including a customizable housing. The different configurations of at least one module within a member may include, for example, modules enclosed in housings, modules directly encapsulated by the member, combination modules including more than one basic module (e.g., control module, power supply module, recharge module), or different geometric arrangements of the modules in the member. Manipulating the virtual IMD 56 may include, for example, positioning virtual IMD 56 at a plurality of locations adjacent virtual skull 54, adjusting the thickness of discrete portions of virtual IMD 56, or the like. In addition, virtual skull 54 may be manipulated, such as forming a recess in a location of skull 54. Once virtual IMD 56 is tailored to the region of virtual skull 54, the dimensions of virtual IMD 56 can be used to construct a physical IMD 64.

For example, the dimensions of virtual IMD 56 may be provided to a computer numerical control (CNC) machine tool, which machines a material into a shape conforming to the dimensions of the virtual IMD 56, as described in further detail below. In other examples, the dimensions may be provided to a CNC machine tool, which machines a form conforming to the dimensions, over which a custom housing is drawn. In yet other examples, the dimensions may be provided to a CNC machine tool, which machines a mold insert to conform to a negative impression of the shape, against which a custom member is molded.

In some examples, determining a shape of the IMD (44) may include identifying a location at which the IMD is to be implanted. In some examples, a surgeon may wish to implant an IMD in a different location for different patients because of, for instance, a different therapy location for one or more of the patients, a feature of a skull or scalp of a patient, or a preference of the surgeon for implanting the IMD proximate to or more spaced from a burr hole through which a lead enters the cranium. For example, some surgeons may prefer to implant the IMD proximate to the burr hole to minimize the distance traversed by the leads, in order to minimize the surgical trauma. Other surgeons may prefer to implant the IMD a greater distance from the burr hole, to minimize the risk of infection in the brain or at the burr hole due to the IMD. Accordingly, a single implantation location may not be preferred for all IMDs or all patients.

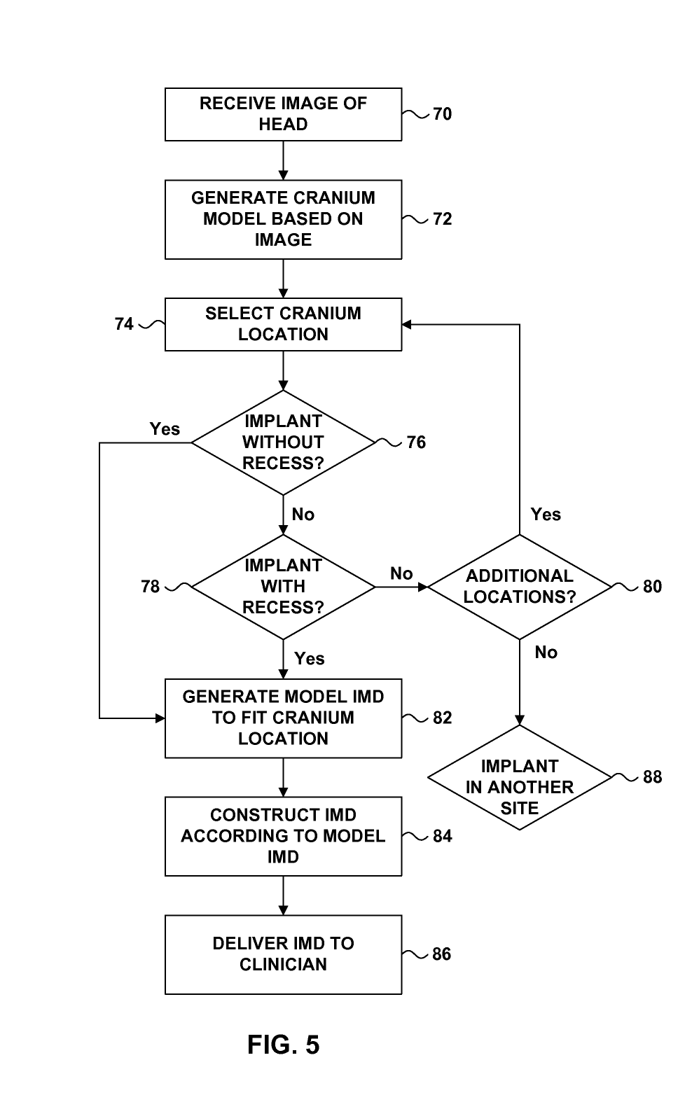

FIG. 5 is a flow diagram illustrating an example technique that includes identifying potential implantation locations for an implanted IMD. Before implanting the implanted device, the surgeon should ordinarily make a determination about what site is appropriate for the patient. Accordingly, the surgeon may direct that the patient be imaged using one or more medical imaging techniques such as X-ray, magnetic resonance imaging (MRI), CT-scan or fluoroscopy. A computer system (e.g., system 52) receives the image(s) of the head (70) and generates a cranium model based on the image(s). A user, such as a surgeon or technician, may select a cranium location at which an implant may be feasible (74). The cranium location may be selected based on a number of considerations, including, for example, a location within the brain of the patient at which one or more leads are to be implanted, a shape or thickness of the cranium of the patient, a condition of the scalp of the patient, a location of vascular structures, or the like.

Before making a cranial implantation, the system, surgeon or technician determines whether the patient is a candidate for a cranial implantation, i.e., whether the cranium of the patient is suitable for such an implantation, considering criteria such as skull curvature, skull thickness, scalp thickness, location of vascular and neurological structures, potential incision locations, and the like. The system, surgeon or technician may determine if cranial implantation without a recess is feasible (76). If the patient has a normal skull and scalp, and has no irregularities that would be expected to impede implantation without a recess, the system can generate a model IMD to fit the cranium location (82).

In some cases, the patient cranial implantation without a recess may not be feasible, but the patient may be a candidate for a cranial implantation with a recess. In these cases, the system, surgeon or technician considers whether a cranial implantation with a recess is indicated (78). When a cranial implantation with a recess is feasible, the system can generate a model IMD to fit the cranium location including the recess (82). In some procedures, the recess may be formed in the cranium by a computer-controlled device.

In some cases, the system, surgeon or technician may determine that the selected cranial location is not suitable for implantation either with or without a cranial recess. The system, surgeon or technician may then determine whether there are additional cranial locations to consider (80). If there are additional cranial locations, the system, surgeon or technician n selects a cranial location (74), determines whether implantation without a recess (76) or with a recess (78) is feasible, and may iterate this process at multiple locations (80), until a suitable location is determined.

In some cases, the system, surgeon or technician may determine that the patient is not a candidate for cranial implantation. In such cases, the surgeon may implant the IMD at another site (88), such as the neck or trunk of the patient.

In examples in which the system, surgeon or technician determines a cranial location at which the IMD may be implanted, a model IMD is generated to fit the cranial location (82). As described above, the model IMD may be a virtual model or a physical model (e.g., a moldable material), and may be tailored to a location in a model of the cranium of the patient corresponding to the cranial location. A physical IMD is then constructed based on the model IMD (84). Constructing the physical IMD may include constructing a custom member or a custom housing, and may include, for example, compression molding, injection molding, machining, drawing, or the like.

In some examples, at least the step of constructing the IMD (84) is performed at a location remote from the surgeon. In these embodiments, the method may include delivering the IMD to the surgeon, or any type of clinician, clinic or medical facility, for implantation in the patient (86). For example, because a custom member or custom housing may be manufactured for each custom IMD, the manufacturing may be more efficiently and cost-effectively performed at a location specialized for this function, rather than at a plurality of manufacturing locations located on-site at the surgeon's place of practice. Accordingly, in some examples, the dimensions of the model IMD may be provided to the manufacturing location, the custom IMD may be constructed, and the custom IMD delivered to the surgeon for implantation in the patient. Accordingly, construction of an IMD to conform to a model IMD or otherwise to conform to shape determined by imaging is different than and may provide advantages relative to on-site, e.g., prior to or during surgery, modification of the IMD, e.g., by manipulation or trimming, by a surgeon or other person at the surgeon's place of practice.

Additionally, in some examples, a computer system (e.g., computer system 52) may automatically determine the shape of the IMD. In some examples, the computer system may also automatically determine the implant location. In other examples, a physician or technician may indicate an implant location and the computer system may automatically determine a shape of the IMD to conform to this location.

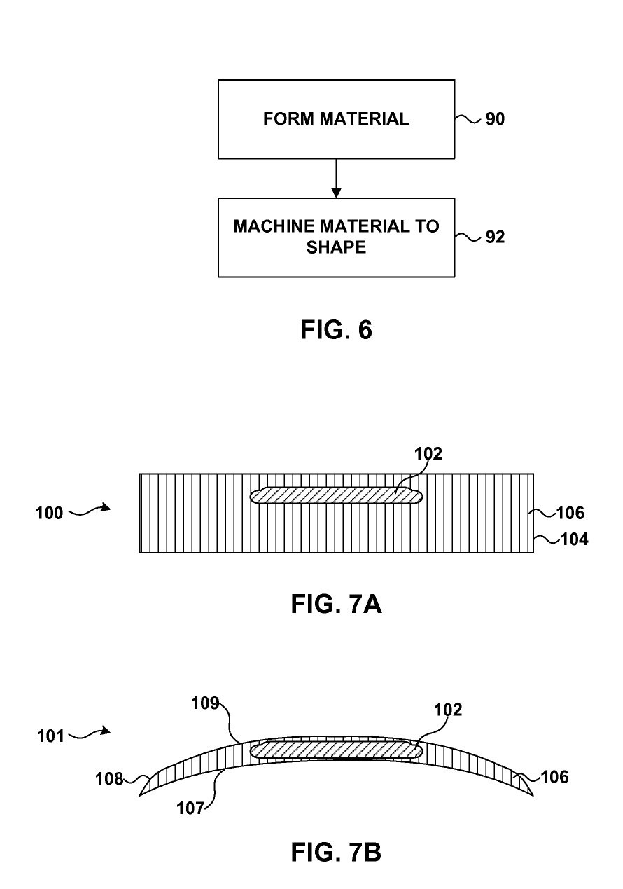

As described briefly above, constructing the IMD (46, 84) may include constructing a custom member or a custom housing to conform to the determined shape, and the custom member and custom housing may be constructed in a plurality of ways. In some examples, as illustrated in FIG. 6, constructing the custom member may include forming a block of material (90) and machining the block of material into custom member that conforms to the shape (92). For example, as shown in a cross-section view in FIG. 7A, an example IMD 100 may initially be formed as a generic block 104 of material 106 around a module 102 encapsulated therein. In some examples, the block may encapsulate more than one module. IMD 100 is a generic IMD, e.g., an IMD that has not yet been customized for a particular patient to conform to a shape, which may be mass produced.

In some examples, module 102 may be enclosed within a hermetic housing. In some examples, the hermetic housing may comprise titanium, stainless steel, or another alloy, such as a titanium alloy, as examples. The hermetic housing may include a surface shaped to generally conform to a curvature of a skull, such as a concave surface. In other examples, the housing may generally be prismatic in shape.

Material 106 may generally comprise a polymer. For example, material 106 may comprise silicone, polyurethane, polysulfone, epoxy, or the like. Block 104 may be molded around module 102, and may form any general shape. For example, block 104 may comprise a rectangular shape, as shown in FIG. 7A, or may comprise a spherical shape, a three-dimensional elliptical shape, or an irregular shape. In some embodiments, block 104 may comprise a shape that approximates the curvature of a typical skull of a patient, such as including a surface comprising a concave shape and an opposite surface comprising a convex shape (similar to the shape of customized IMD 101 in FIG. 7B).

When the shape of an IMD is determined based on an image of a head of a patient into which the IMD is to be implanted, the block 104 of material of generic IMD 100 may be machined into the desired shape 108, forming a customized IMD 101, as shown in FIG. 7B. In some examples, the machining is performed by a CNC machine tool. Customized IMD 101 may include a first surface 107 that is generally concave and a second surface 109 that is generally convex. The specific shapes of first surface 107 and, optionally, second surface 109 may be determined based on the image of the skull of the patient (and scalp), and the region at which IMD 101 is to be implanted. For example, first surface 107 and/or second surface 109 may include deviations from concavity and convexity, respectively, including, for example, notches, grooves, protrusions, concave or convex sections with different radii of curvature, planar sections, or the like, which may improve the extent to which first surface 107 conforms to the location of the skull at which IMD 101 is to be implanted.

The extent to which custom member 108 extends beyond module 102 is merely an example. In some examples, it may desirable to minimize the total volume of customized IMD 101 to reduce the amount of tension on the scalp of the patient. Accordingly, in some examples, custom member 108 may extend only a small length beyond the edges of module 102.

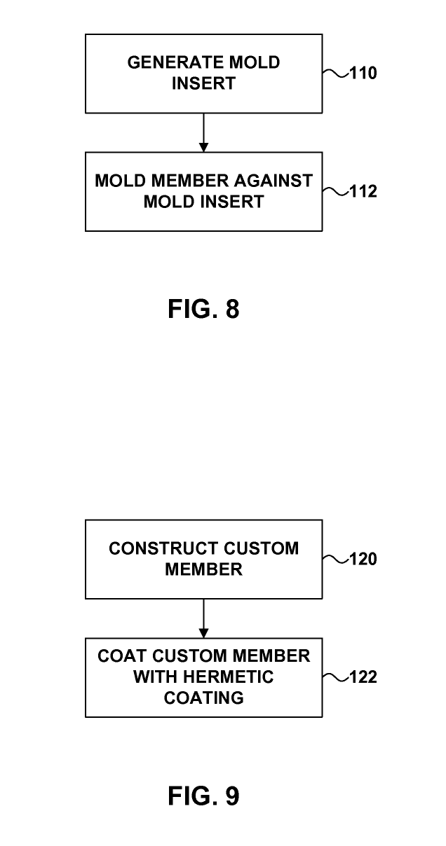

FIG. 8 illustrates another example method of constructing the custom member. In the example of FIG. 8, a mold insert is first generated (110). The mold insert may correspond to the shape of a section of a skull of a patient. As described above, the mold insert may comprise a section of a physical model of the skull of the patient, or a mold insert formed based on a physical or virtual model of the skull of the patient and the IMD.

A material of which the custom member is comprised is then molded against the mold insert (112). The material may comprise, for example, silicone, polyurethane, epoxy, or polysulfone. In some examples, the material already encloses any modules to be at least partially encapsulated by the custom member. In other examples, the custom member may be molded including a cavity into which the module(s) (including or not including a housing that encloses the module(s)) may be inserted. The custom member may then be positioned over the module(s) and attached using, for example, an adhesive or sutures.

In many examples, the at least one module of the IMD may be hermetically sealed to prevent contamination and/or corrosion of the components in the module by bodily fluids, or to prevent adverse effects to the patient from a non-biocompatible material in the module. As described above, in some examples the at least one module is enclosed in hermetic housing.

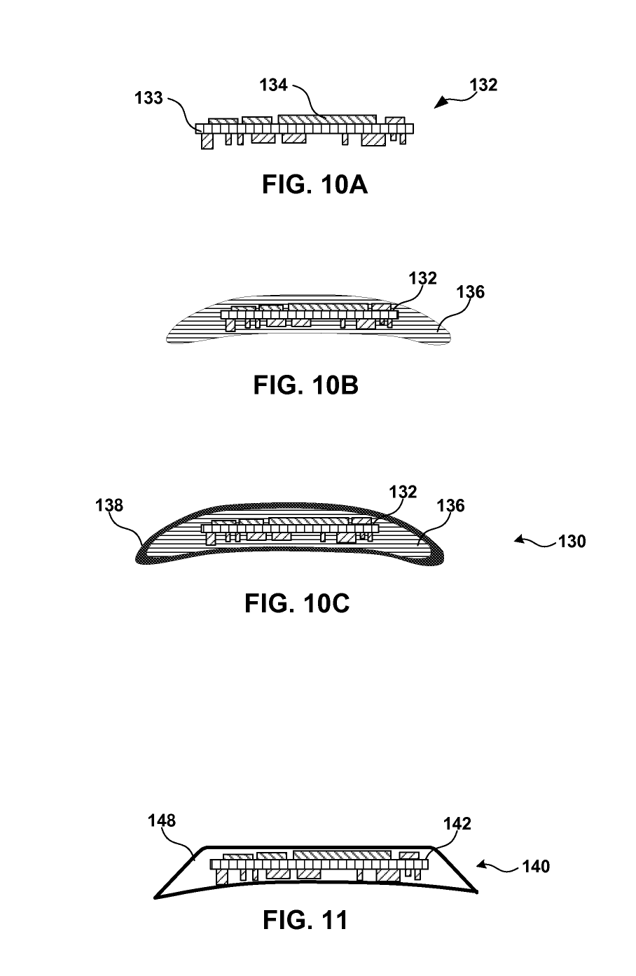

In some examples, constructing the IMD may include coating a custom member with a hermetic coating, as illustrated in FIGS. 9 and 10 A-C. The custom member may be constructed (120) by machining a preformed block of material or may be formed by molding material against a mold insert, as described above. In FIGS. 10A and 10B, the custom member 136 is molded over a module 132.

The custom member 136 may then be coated with a hermetic coating 138 (122), such as, for example, titanium alloy, or gold, as shown in FIG. 10C. The hermetic coating 138 may be applied using a number of processes including, for example sputtering, chemical vapor deposition, physical vapor deposition, laser microdeposition, or the like. The hermetic coating 138 may be a relatively thin film that conforms to the shape of the custom member 136.

Hermetic coating 138 may facilitate embodiments in which module 132 does not include a housing, as illustrated in FIGS. 10A-10C. In the example illustrated by FIG. 10A, module 132 includes a circuit board 133, which may be a hybrid circuit board. Circuit board 133 carries a variety of components 134, which may include, as examples, one or more processors or other control electronics, such as microprocessors, application specific integrated circuits, or other integrated circuits, digital or analog circuitry, a power source such as a rechargeable or nonrechargeable battery, communication circuitry, an antenna, electrical stimulation generation circuitry, a pump, a reservoir, or physiological parameter sensing circuitry. Some examples may include more than one circuit board 133. Further, in some examples, module 132 may include a hermetic or nonhermetic housing to, for example, protect circuit board 133 and components 134 from forces or exposure to harmful materials or conditions during construction of custom member 136 or coating 138.

As illustrated in FIG. 11, constructing the IMD may also include forming a custom housing. For example, the IMD 140 may include at least one module 142, which may be substantially similar to module 132 described with reference to FIGS. 10A-10C, enclosed by custom housing 148. In some examples, the at least one module 142 may be enclosed in a standard housing, which is then enclosed by the custom housing 148. The standard housing may be generally prismatic in shape, or may include a contoured shape, such as a concave or convex surface.

The custom housing 148 may be formed by drawing a metal over a form. For example, the dimensions of the form may be determined by a computer system (e.g., computer system 52) and the form may be constructed by a CNC machine tool, or by molding a material against a physical model of the skull of a patient, as described above. The custom housing may comprise, for example, titanium, stainless steel, or an alloy, such as, for example, a titanium alloy.

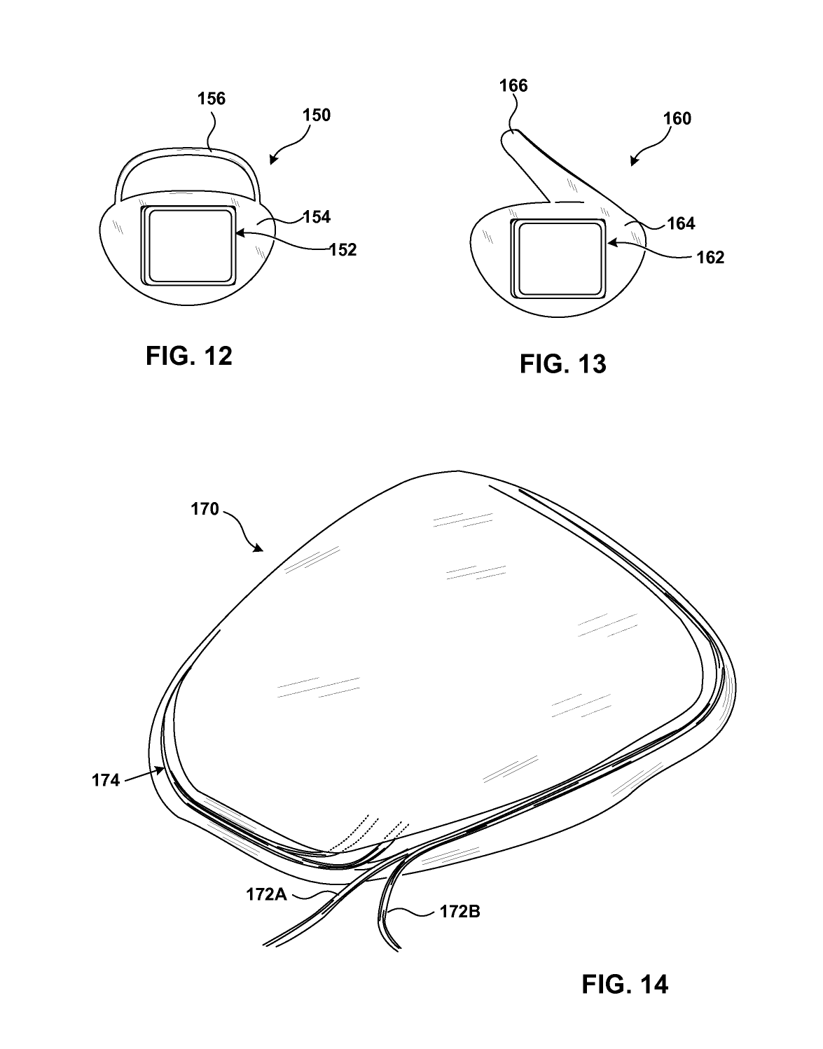

As shown in FIGS. 12 and 13, constructing the IMD may also include forming other custom features in the custom member or custom housing. For example, the custom member 154 may include a grippable access structure formed in or coupled to member 154. As illustrated in FIG. 12, IMD 150 may include one or more modules 152 that are at least partially encapsulated by custom member 154. In some examples at least one of the modules is enclosed in a housing. In addition, IMD 150 includes a grippable access structure coupled to member 154, in the form of a loop 156. Loop 156 can be formed integral with the member or may be mechanically coupled to the member. Loop 156 can be dimensioned such that a surgeon may grip loop 156 with an instrument such as a forceps, or the surgeon has the option to grip loop 156 with his fingers. Loop 156 may include a wire or other radiopaque element (not shown) that would make loop 156 visible during imaging.

FIG. 13 illustrates another example IMD formed with a custom member. IMD 160 is a low-profile IMD that includes one or more modules 162 with housings that are at least partially encapsulated by a custom member 164. In addition, IMD 160 includes a grippable access structure coupled to custom member 164, in the form of a tab 166. Like loop 156 in FIG. 12, tab 166 can be formed integral with the member or may be mechanically coupled to the member, and can be dimensioned to give the surgeon flexibility to grip the structure by hand or with an instrument. In some examples, tab 166 may include a radiopaque marker that would make tab 166 visible during imaging.

While FIGS. 12 and 13 illustrate custom grippable access structures including a loop and a tab coupled to or formed in a custom member, the custom grippable access structure may also include, for example, a handle or handle-like structure formed in the custom member. In some examples, the custom grippable access structure may also a handle, loop, tab, aperture, slot, or the like formed in or coupled to a custom housing. In some examples, the grippable access structure may be provided facilitate retrieval of the IMD by a surgeon in the event the IMD (e.g., IMD 150 or 160) is explanted. The form of the grippable access structure may be based on a preferred retrieval method indicated a surgeon who implants and may explant the IMD.

As described briefly above, the custom member or custom housing may also include custom lead management features. For example, FIG. 14 is a perspective view of an embodiment of a low-profile IMD 170 that includes a lead management structure. IMD 170 includes one or more modules (not shown) within housings and a member that at least partially encapsulates the housings. IMD 170 is configured to be implanted between a scalp and a skull of a patient.

Leads 172A and 172B (collectively “leads 172”) are deployed around IMD 170 in a lead management structure. A lead management structure is a structure in IMD 170 that is configured to receive and protect the bodies of leads 172 that are coupled to the IMD 170. In particular, a lead management structure is a structure that is configured to receive and protect the bodies of the leads as opposed to the terminals of the leads by, for example, minimizing the stress experience by the leads. Lead management structures include, but are not limited to, structures that route, fixate or anchor the lead bodies. Examples of a lead management structures include a groove or a cavity that receives a lead body.

One of the practical problems associated with the leads is that the leads can be difficult to manage. The leads can twist, bend, slide and otherwise move. The propensity of leads to move can be inconvenience during implantation, and can also be a problem during explantation. If the leads move after implantation, there is an increased risk of damage to leads during explantation.

In FIG. 14, the lead management structure is a groove 174 formed in a member or housing of IMD 170, and leads 172A and 172B are wrapped around IMD 170 in groove 174. The dimensions of the groove may be a function of the length of the leads and the dimensions of IMD. The placement of groove 174 around the periphery of IMD 170 is for illustrative purposes, and the lead management structure is not limited to the particular lead management structure shown in FIG. 14.

The lead management structure need not be formed in member 174. In some examples, the lead management structure can be constructed of a separate material, such as a protective material that would resist damage in the event the incision should cut across IMD 170. Cut-resistant materials include, but are not limited to, metals and materials including embedded wire or polymer meshes. Furthermore, the lead management structure need not be located around the periphery as shown in FIG. 14, but in some embodiments can be located underneath member 174 and modules 172. Lead management structures can not only direct lead bodies around IMD 170, but can direct the lead bodies over or under IMD 170. In other examples, the lead management structure may include a cavity formed in a custom member, or may include a groove or cavity formed in or coupled to a custom housing.

In addition, the custom member or custom housing may include a custom position at which at least one lead exits the IMD. For example, the custom position may be determined based on a location of a burr hole and an orientation of the IMD when implanted adjacent a patient's skull. In some examples, the custom position may be chosen so that the at least one lead exits the IMD proximate the burr hole. In other examples, the custom position may be determined based on a position indicated by a surgeon.

Various examples have been described. However, one of ordinary skill in the art will appreciate that various modifications may be made to the described examples. For example, although described herein primarily with reference to construction of a custom IMD for implantation within the head and, more particularly, on the skull beneath the scalp, the techniques described herein may be used to create a custom IMD for any therapeutic or monitoring function, and for implantation in any location within a patient. Furthermore, implantation of a cranial IMD is not limited to implantation on top of the head as illustrated herein.

1. A method comprising:
receiving an image of tissue of a patient;
determining a shape of a custom member that at least partially encapsulates a module of an implantable medical device based on the image, the module comprising control electronics; and
constructing the custom member to conform to the shape.

2. The method of claim 1, wherein the implantable medical device comprises a housing that encloses the module, and wherein the custom member at least partially encapsulates the housing.

3. The method of claim 2, wherein constructing the custom member comprises molding the custom member over at least a portion of the housing.

4. The method of claim 2, wherein constructing the custom member comprises:
forming the custom member as a block of material; and
machining the block of material to conform to the shape.

5. The method of claim 2, wherein the housing is hermetic.

6. The method of claim 5, wherein the custom member comprises at least one of silicone, polysulfone, or polyurethane.

7. The method of claim 1, further comprising coating the member with a hermetic coating.

8. The method of claim 7, wherein constructing the custom member comprises molding the custom member.

9. The method of claim 7, wherein constructing the custom member comprises:
forming the custom member as a block of material; and
machining the block of material to conform to the shape.

10. The method of claim 7, wherein the custom member comprises at least one of polysulfone or epoxy.

11. The method of claim 7, wherein coating the custom member comprises coating the member with at least one of titanium or a titanium alloy.

12. The method of claim 11, wherein coating the custom member with titanium comprises sputtering at least one of titanium or a titanium alloy on the custom member.

13. The method of claim 1, wherein receiving the image comprises receiving an image generated by at least one of X-ray, magnetic resonance imaging, CT-scan, or fluoroscopy.

14. The method of claim 1, wherein receiving the image comprises receiving an image of at least one of a head or a skull.

15. The method of claim 1, further comprising delivering the implantable medical device including the custom member to at least one of a clinic, a clinician, or a surgeon.

16. A method comprising:
receiving an image of tissue of a patient;
determining a shape of a custom housing of an implantable medical device comprising control electronics based on the image; and
constructing the custom housing to conform to the shape.

17. The method of claim 16, wherein receiving the image comprises receiving an image generated by at least one of X-ray, magnetic resonance imaging, CT-scan, or fluoroscopy

18. The method of claim 16, wherein receiving the image comprises receiving an image of at least one of a head or a skull of the patient.

19. The method of claim 16, further comprising delivering the implantable medical device including the custom housing to at least one of a clinic, a clinician, or a surgeon.

20. The method of claim 16, wherein constructing the custom housing comprises constructing the custom housing from at least one of titanium or a titanium alloy.

21. The method of claim 16, wherein constructing the custom housing comprises drawing a metal over a form that conforms to the shape.

22. An implantable medical device comprising:
a module comprising control electronics;
a custom member that at least partially encapsulates the module, wherein the custom member is constructed to conform to a shape determined based on an image of tissue of a patient in which the implantable medical device is to be implanted; and
a hermetic coating formed over the custom member.

23. The implantable medical device of claim 22, wherein the hermetic coating comprises at least one of titanium or a titanium alloy.

24. The implantable medical device of claim 22, wherein the custom member comprises polysulfone.

25. The implantable medical device of claim 22, wherein the custom member comprises epoxy.

26. The implantable medical device of claim 22, wherein the custom member fully encapsulates the module.

27. An implantable medical device comprising a custom housing for housing a module comprising control electronics, wherein the custom housing is constructed to conform to a shape determined based on an image of tissue of a patient in which the implantable medical device is to be implanted.

28. The implantable medical device of claim 27, wherein the custom housing comprises at least one of titanium or a titanium alloy.

29. The implantable medical device of claim 27, wherein the custom housing forms a hermetic enclosure.

30. The implantable medical device of claim 27, wherein the module is enclosed in a standard housing, and wherein the custom housing encloses the standard housing.
